(19) United States
(12) Patent Application Publication
(10) Pub. No.: US 2023/0149663 A1
(43) Pub. Date: May 18, 2023

(54) STEERABLE MEDICAL DEVICE, HANDLE FOR A MEDICAL DEVICE, AND METHOD FOR OPERATING A MEDICAL DEVICE

(71) Applicant: Boston Scientific Medical Device Limited, Galway (IE)

(72) Inventors: James Klein, Mississauga (CA); Jan-Hung Chen, Mississauga (CA)

(21) Appl. No.: 18/151,328

(22) Filed: Jan. 6, 2023

Related U.S. Application Data
(63) Continuation of application No. PCT/IB2021/056053, filed on Jul. 6, 2021.
(60) Provisional application No. 63/049,193, filed on Jul. 8, 2020.

(51) Int. Cl. A61M 25/01; A61B 1/005; A61B 17/00

(52) U.S. Cl. CPC A61M 25/0136; A61B 1/0052; A61B 17/00234; A61M 25/0147; A61B 2017/00309; A61M 2025/015

(57) ABSTRACT

A handle for a medical device includes a rotatable knob assembly and a slide assembly. The knob assembly is couplable to the slide assembly to drive movement of the slide assembly by rotation of the knob assembly. A slip clutch assembly is between the knob and slide assemblies. The slip clutch assembly includes a first disc and a second disc. The first disc is secured to the knob assembly and is rotatable with the knob assembly. The second disc is configured to transmit rotation to the slide assembly. The first disc and/or the second disc is translatable between a first position and a second position. In the first position, the first and second discs are moved into engagement to couple the rotatable knob to the slide assembly. In the second position, the first and second discs are moved out of engagement to decouple the rotatable knob from the slide assembly.

### CROSS REFERENCE TO RELATED APPLICATION

This application is a continuation application of International Application No. PCT/IB2021/056053, filed Jul. 6, 2021, titled “STEERABLE MEDICAL DEVICE, HANDLE FOR A MEDICAL DEVICE, AND METHOD FOR OPERATING A MEDICAL DEVICE,” which claims priority to U.S. Provisional Application No. 63/049,193, filed Jul. 8, 2020, titled “STEERABLE MEDICAL DEVICE, HANDLE FOR A MEDICAL DEVICE, AND METHOD FOR OPERATING A MEDICAL DEVICE,” the entire disclosures of which are incorporated herein by reference.

### FIELD

This document relates to medical devices. More specifically, this document relates to steerable medical devices such as steerable sheaths, catheters, and introducers.

### SUMMARY

The following summary is intended to introduce the reader to various aspects of the detailed description, but not to define or delimit any invention.

Steerable medical devices are disclosed. According to some aspects, a steerable medical device includes a handle having a rotatable knob assembly and housing a slide assembly. The knob assembly is couplable to the slide assembly to drive movement of the slide assembly by rotation of the knob assembly. An elongate tool extends from the handle. At least one control wire is coupled between the slide assembly and the tool. Movement of the slide assembly causes tensioning of the control wire, and tensioning of the control wire causes deflection of the tool. A first catch is coupled to the knob assembly, and a second catch is coupled to the slide assembly. When torque applied to the knob assembly is below a threshold value, the first catch grips the second catch to drive movement of the slide assembly by rotation of the knob assembly. When torque applied to the knob assembly is above the threshold value, the first catch releases the grip on second catch to decouple the knob assembly from the slide assembly, to avoid movement of the slide assembly by rotation of the knob assembly.

In some examples, the first catch includes a first set of spaced apart protrusions arranged annularly around an axis of rotation and extending generally parallel to the axis of rotation, and the second catch includes a second set of spaced apart protrusions arranged annularly around the axis of rotation and extending generally parallel to the axis of rotation. The protrusions of the first set can be removably receivable between the protrusions of the second set.

In some examples, the first set of protrusions extend from a first hub, and the first hub is fixed to the knob assembly and rotatable with the knob assembly. The second set of protrusions can extend from a second hub, and the second hub can be fixed to the slide assembly and rotatable with the slide assembly.

In some examples, when torque applied to the knob assembly is below the threshold value, the protrusions of the first set are received between the protrusions of the second set to grip the second catch with the first catch and thereby drive movement of the slide assembly by rotation of the knob assembly. When torque applied to the knob assembly is above the threshold value, the protrusions of the first set can deflect to clear the protrusions of the second set and release the grip of the first catch on the second catch, and avoid movement of the slide assembly by rotation of the knob assembly. The protrusions can deflect radially outwardly from the axis of rotation. The protrusions of the first set and the protrusions of the second set can include inclined side surfaces, to facilitate deflection of the protrusions of the first set.

In some examples, the second catch includes a shaft extending along the axis of rotation, and the shaft abuts the first hub.

In some examples, the protrusions of the first set are in the form of resiliently flexible prongs, and the protrusions of the second set are in the form of inflexible bumps defining slots therebetween for receipt of the prongs.

In some examples, the first catch grips the second catch with magnetic forces and/or frictional forces.

In some examples, the tool is a sheath, a catheter, or an introducer.

Handles for medical devices are also disclosed. According to some aspects, a handle for a medical device includes a rotatable knob assembly and a slide assembly. The knob assembly is couplable to the slide assembly to drive movement of the slide assembly by rotation of the knob assembly. A first catch is coupled to the knob assembly, and a second catch is coupled to the slide assembly. When torque applied to the knob assembly is below a threshold value, the first catch grips the second catch to drive movement of the slide assembly by rotation of the knob assembly. When torque applied to the knob assembly is above the threshold value, the first catch releases the grip on the second catch to decouple the knob assembly from the slide assembly to avoid movement of the slide assembly by rotation of the knob assembly.

In some examples, the first catch includes a first set of spaced apart protrusions arranged annularly around an axis of rotation and extending generally parallel to the axis of rotation, and the second catch includes a second set of spaced apart protrusions arranged annularly around the axis of rotation and extending generally parallel to the axis of rotation. The protrusions of the first set can be removably receivable between the protrusions of the second set. The protrusions of the first set are can be the form of resiliently flexible prongs, and the protrusions of the second set can be in the form of inflexible bumps defining slots therebetween for receipt of the prongs.

In some examples, the first set of protrusions extends from a first hub, and the first hub is fixed to the knob assembly and rotatable with the knob assembly. The second set of protrusions can extend from a second hub, and the second hub can be fixed to the slide assembly and rotatable with the slide assembly. The second hub can include a shaft extending along the axis of rotation, and the shaft can abut the first hub.

In some examples, when torque applied to the knob assembly is below the threshold value, the protrusions of the first set are received between the protrusions of the second set to grip the second catch with the first catch and thereby drive movement of the slide assembly by rotation of the knob assembly. In some examples, when torque applied to the knob assembly is above the threshold value, the protrusions of the first set deflect to clear the protrusions of the second set and release the grip on the second catch and avoid movement of the slide assembly by rotation of the knob assembly. The protrusions can deflect radially outwardly from the axis of rotation. The protrusions of the first set and the protrusions of the second set can have inclined side surfaces, to facilitate deflection of the protrusions of the first set.

In some examples, the first catch grips the second catch with magnetic forces and/or frictional forces.

Methods for operating medical devices are also disclosed. According to some aspects, a method for operating a medical device includes: a. with a first catch releasably gripping a second catch, and with the first catch coupled to a knob assembly and the second catch coupled to a slide assembly, applying torque to the knob assembly to rotate the knob assembly; and b. transmitting rotation of the knob assembly to the slide assembly via the first catch and the second catch.

In some examples, the first catch includes a first set of spaced apart protrusions arranged annularly around an axis of rotation and extending generally parallel to the axis of rotation, the second catch includes a second set of spaced apart protrusions arranged annularly around the axis of rotation and extending generally parallel to the axis of rotation, and the protrusions of the first set are removably receivable between the protrusions of the second set. Step b. can include transmitting rotation of the knob assembly to the first set of protrusions to force the first set of protrusions to rotate around the axis of rotation, and transmitting the rotation of the first set of protrusions to the second set of protrusions to force the second set of protrusions to rotate around the axis of rotation.

In some examples, the method further includes: c. increasing the torque applied to the knob assembly to exceed a threshold value; and d. as a result of step c., releasing the grip of the first catch on the second catch.

In some examples, the first catch includes a first set of spaced apart protrusions arranged annularly around an axis of rotation and extending generally parallel to the axis of rotation, the second catch includes a second set of spaced apart protrusions arranged annularly around the axis of rotation and extending generally parallel to the axis of rotation, and the protrusions of the first set are removably receivable between the protrusions of the second set. step d. comprises deflecting the protrusions of the first set to clear the protrusions of the second set. Step d. can include deflecting the protrusions radially outwardly.

In some examples, the method further includes: e. decreasing the torque applied to the knob assembly to fall below the threshold value; and f., as a result of step e., reapplying the grip of the first catch on the second catch.

In some examples, the first catch includes a first set of spaced apart protrusions arranged annularly around an axis of rotation and extending generally parallel to the axis of rotation, the second catch includes a second set of spaced apart protrusions arranged annularly around the axis of rotation and extending generally parallel to the axis of rotation, and the protrusions of the first set are removably receivable between the protrusions of the second set. Step f. can include deflecting the protrusions of the first set to be positioned between the protrusions of the second set.

In some examples, the method further includes providing tactile and/or auditory feedback to indicate that the first catch has released the grip on the second catch, and/or providing a visual indication that the first catch has released the grip on the second catch.

In some examples, step a. includes using magnetic forces and/or frictional forces to grip the second catch with the first catch.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical device;

FIG. 2 is a cutaway plan view of the handle of the medical device of FIG. 1;

FIG. 3A is an enlarged plan view of a slip clutch assembly of the handle of FIG. 2 showing coupling of a knob assembly coupled to a slide assembly by the slip clutch assembly;

FIG. 3B is a longitudinal cross section taken through the slip clutch assembly of FIG. 3A

FIG. 4A is an enlarged plan view similar to that of FIG. 3A, showing decoupling of the knob assembly from the slide assembly by the slip clutch assembly;

FIG. 4B is a longitudinal cross-section taken through the slip clutch assembly of FIG. 4A;

FIG. 5 is an enlarged plan view similar to that of FIG. 4, showing recoupling of the knob assembly to the slide assembly by the slip clutch assembly;

FIG. 6 is a partial exploded view of the knob assembly, slip clutch assembly, and slide assembly of the handle of FIG. 2; and

FIG. 7 is a bottom view of the first catch of the slip clutch assembly of FIG. 7; and

FIG. 8 is a top view of the second catch of the slip clutch assembly of FIG. 7.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are steerable medical devices that include a handle and a tool such as a sheath, a catheter, or an introducer. The handle can enable the user to manipulate or steer the tool in a desired direction. More specifically, the handle can include a knob assembly that is rotatably coupled to a housing of the handle. In operation, the rotation of the knob assembly in a first rotational direction can allow the user to steer or deflect the tool in a first direction, whereas the rotation of the knob assembly in a second rotational direction can allow the user to steer or deflect the tool in a second direction. The rotation of the knob can be converted into a deflection of the tool via a slide assembly, which can be within the housing, and one or more control wires, which are connected between the slide assembly and the tool. The slide assembly can include a threaded shaft (also referred to as a bolt), and a slider (also referred to as a carriage) that is slidable on the threaded shaft. Rotation of the knob can cause rotation of the threaded shaft, which can cause linear translation of the slider along the threaded shaft. This translation of the slider causes a tensioning of the control wire(s), which results in a deflection of the tool. For simplicity, details of the slider and the control wires are not disclosed herein. However, related sliders and control wires are disclosed in, for example, U.S. Pat. No. 10,661,057 (Davies et al.), which is incorporated herein by reference in its entirety. Furthermore, steerable medical devices including sliders and control wires are sold Baylis Medical Company, Inc. (Montreal, Canada) under the brand name SureFlex® Steerable Guiding Sheath.

The steerable medical devices disclosed herein are configured to avoid or prevent or reduce the risk of failure due to excessive tension being applied to the control wire(s). Such excessive tension can occur, for example, if a stiff secondary tool is within the tool, preventing deflection of the tool, but a user attempts to force rotation of the knob assembly. Such excessive tension can further occur if the tool is caught on patient anatomy and thus cannot deflect, but a user attempts to force rotation of the knob assembly. Such excess tension can further occur if force is applied to the tool without rotating the knob, for example if the tool is pressed by patient anatomy.

In the devices disclosed herein, to avoid or prevent or reduce the risk of failure due to excessive tension being applied to the control wire(s), a slip clutch assembly is provided between the knob assembly and the slide assembly. In use, when a relatively low amount of tension is on the control wires and torque is applied to the knob assembly (i.e. torque of below a threshold value), the knob assembly rotates, and rotation of the knob assembly causes rotation of the slide assembly via the slip clutch assembly, which couples the knob assembly and the slide assembly; however, when the amount of tension on the control wires is relatively high and torque is applied to the knob assembly (i.e. torque of above the threshold value), the slip clutch assembly decouples the knob assembly from the slide assembly, to avoid rotation of the slide assembly with the knob assembly, and thereby avoid additional tension from being applied to the control wires. This can in turn avoid or prevent or reduce the risk of breaking of the control wires or disconnection of the joint between the control wires and the slide mechanism and/or tool. Furthermore, this can avoid or prevent or reduce the risk of tissue damage if the tool is caught on patient anatomy.

As used herein, the term “slip clutch assembly” refers to an assembly that couples a first element (e.g. a knob assembly) to a second element (e.g. a slide assembly) to transmit rotation from the first element to a second element when the torque applied to the first element is below a threshold value, and decouples the first element and the second element to prevent transmission of rotation from the first element to the second element when the torque applied to the second element is above a threshold value.

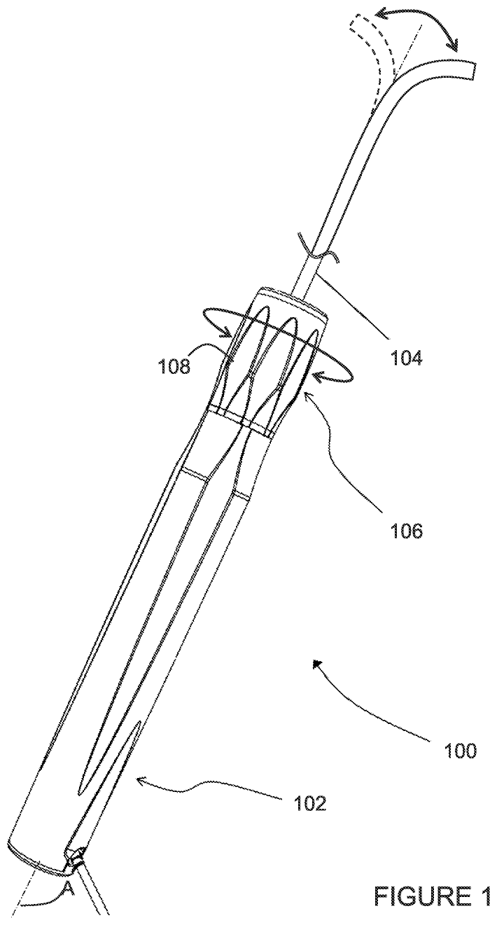

Referring now to FIG. 1, an example steerable medical device 100 is shown. The steerable medical device 100 generally includes a handle 102 and an elongate tool 104 extending from the handle. The tool 104 can be, for example (but not limited to), a sheath, a catheter, or an introducer. The handle 102 includes a rotatable knob assembly 106, which can be rotated about an axis of rotation A to steer the tool 104. Rotation of the knob assembly 106 in a first direction (e.g. clockwise) can cause the tool 104 to deflect in a first direction (i.e. to the configuration shown in dotted line in FIG. 1), and rotation of the knob assembly 106 in a second direction (e.g. counter-clockwise) can cause the tool 104 to deflect in a second direction (i.e. back to the configuration shown in solid line in FIG. 1).

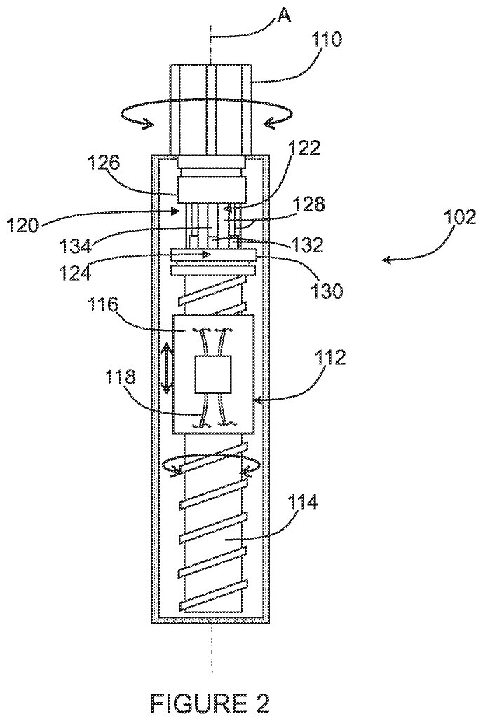

Referring still to FIG. 1 and also to FIG. 2, the knob assembly 106 includes an outer knob 108 (shown in FIG. 1), which is grasped and manipulated by the user, and an inner knob 110 (shown in FIG. 2) which is rotated by rotation of the outer knob 108.

Referring still to FIG. 2, a slide assembly 112 is housed within the handle 102. As will be described in further detail below, the knob assembly 106 (only the inner knob 110 of which is shown in FIG. 2) is couplable to the slide assembly 112 to drive movement of the slide assembly 112 by rotation of the knob assembly 106. More specifically, the slide assembly 112 includes a threaded shaft 114 and a slider 116 that is received on the threaded shaft 114 and is translatable along the threaded shaft 114. When the knob assembly 106 is coupled to the slide assembly 112, rotation of the knob assembly 106 causes rotation of the threaded shaft 114 about the axis of rotation A. Rotation of the threaded shaft 114 causes translation of the slider 116 along the threaded shaft 114. A control wire 118 is in turn coupled between the slider 116 of the slide assembly 112 and the tool 104 (shown in FIG. 1). Movement of the slide assembly 112—i.e. translation of the slider 116 caused by rotation of the threaded shaft 114—causes tensioning of the control wire 118, and tensioning of the control wire 118 causes deflection of the tool 104. As mentioned above, details of the slider 116 and control wire 118, and the connection between the slider 116, control wire 118 and tool 104 are disclosed in U.S. Pat. No. 10,661,057 (Davies et al.), and are not repeated herein.

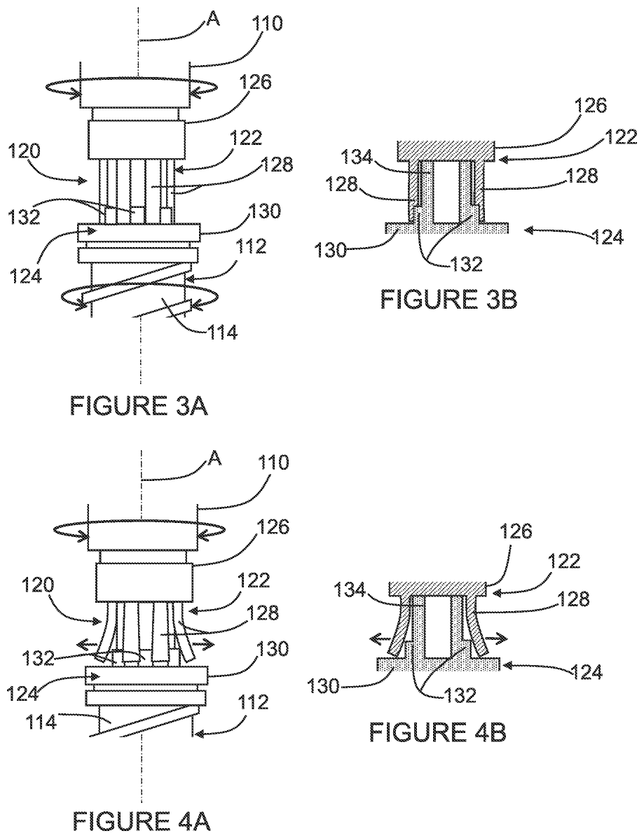

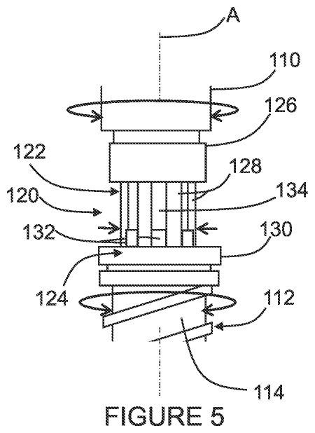

Referring to FIGS. 3 to 5, a slip clutch assembly 120 is between the knob assembly 106 (only the inner knob 110 of which is shown in FIGS. 3 to 5) and the slide assembly 112. As shown in FIGS. 3A and 3B and as will be described in more detail below, when torque applied to the knob assembly 106 is below a threshold value (e.g. when the control wire 118, not shown in FIGS. 3A to 5, is under a relatively low amount of tension, as would occur during routine use), the slip clutch assembly 120 couples the inner knob 110 of the knob assembly 106 to the threaded shaft 114 of the slide assembly 112 to drive movement of the slide assembly 112 by rotation of the knob assembly 106. As shown in FIGS. 4A and 4B and as will be described in more detail below, when torque applied to the knob assembly 106 is above a threshold value (e.g. when the control wire 118 is under relatively high tension and rotation of the knob assembly 106 is continued), the slip clutch assembly 120 decouples the inner knob 110 of the knob assembly 106 from the threaded shaft 114 of the slide assembly 112 to prevent movement of the slide assembly 112 by rotation of the knob assembly 106. This in turn prevents further tensioning of the control wire 118, which in turn avoids or prevents or minimizes the risk of failure. As shown in FIG. 5, when the torque applied to knob assembly 106 is lowered to below threshold value, the slip clutch assembly 120 recouples the inner knob 110 of the knob assembly 106 to the threaded shaft 114 of the slide assemblyl12, to again drive movement of the slide assembly 112 by rotation of the knob assembly 106.

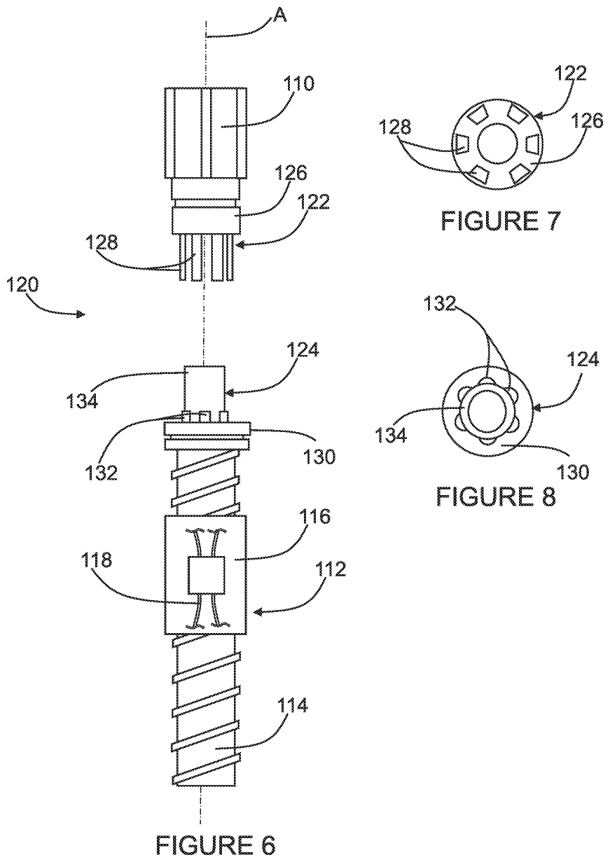

Referring to FIG. 6, in the example shown, the slip clutch assembly 120 includes a first catch 122 that is coupled to the knob assembly 106 (only the inner knob 110 of which is shown in FIG. 6) and that is rotatable with the knob assembly 106, and a second catch 124 that is coupled to the slide assembly 112 and that is rotatable with the slide assembly 112. When the torque applied to the knob assembly 106 is below a threshold value, the first catch 122 grips the second catch 124 to drive movement of the slide assembly 112 by rotation of the knob assembly 106 (as shown in FIGS. 3A, 3B, and 5). When torque applied to the knob assembly 106 is above the threshold value, the first catch 122 releases the grip on second catch 124 to decouple the knob assembly 106 from the slide assembly 112, to avoid movement of the slide assembly 112 by rotation of the knob assembly 106 (as shown in FIGS. 4A and 4B).

More specifically, referring still to FIG. 6 and also to FIG. 7, in the example shown, in order to provide releasable gripping, the first catch 122 includes a first hub 126 that is fixed to the inner knob 110 of the knob assembly 106, and a first set of protrusions 128 (only two of which are labelled) that extend from the first hub 126. The protrusions 128 are in the form of prongs that are spaced apart, arranged annularly around the axis of rotation A, and extend generally parallel to the axis of rotation A. As can be seen in FIG. 7, the protrusions 128 have inclined side surfaces—i.e. in the example shown the protrusions 128 are trapezoidal in cross section, with the short side of each trapezoid facing radially inwardly. Furthermore, the protrusions 128 are resiliently flexible, and can deflect (or bend or flex) radially away from the axis of rotation A when lateral force is applied thereto (as shown in FIGS. 4A and 4B), and will snap back towards the axis of rotation A when the force is released.

Referring still to FIG. 6 and also to FIG. 8, the second catch 124 includes a second hub 130 that is fixed to the threaded shaft 114 of the slide assembly 112. A shaft 134 extends from the second hub 130, and abuts the first hub 126. A second set of protrusions 132 (only two of which are labelled) extend longitudinally from the second hub 130 and radially from the shaft 134. The protrusions 132 are in the form of relatively inflexible bumps that are arranged annularly around the axis of rotation A and extend generally parallel to the axis of rotation A. The protrusions 132 have inclined side surfaces—i.e. in the example shown the protrusions 128 are semi-circular in cross section, with the wide side of each semi-circle facing radially inwardly and the rounded side facing radially outwardly. The protrusions 132 define slots therebetween, and the protrusions 128 of the first catch 122 are removably receivable in the slots. That is, the protrusions 128 are positioned between the protrusions 132, to form an interleaved arrangement of the protrusions 128 of the first catch 122 and the protrusions 132 of the second catch 124.

Referring back to FIGS. 3A and 3B, when the torque applied to the knob assembly 106 is below the threshold value (as would occur during routine use), the protrusions 128 remain between the protrusions 132. This interleavement of the protrusions 128 of the first catch 122 and the protrusions 132 of the second catch 124 results in the first catch 122 gripping the second catch 124, to drive movement of the slide assembly 112 (i.e. rotation of the threaded shaft 114) by rotation of the knob assembly 106. That is, rotation of the knob assembly 106 is transmitted to the slide assembly 112 via the first catch 122 and the second catch 124. Rotation of the knob assembly 106 is transmitted to the first hub 126 to cause the first hub 126 to rotate; rotation of the first hub 126 is transmitted to first set of protrusions 128 to cause the first set of protrusions 128 to rotate; rotation of the first set of protrusions 128 is transmitted to the second set of protrusions to force the second set of protrusions 132 to rotate; rotation of the second set of protrusions 132 is transmitted to the second hub 130 to force the second hub 130 to rotate; and rotation of the second hub 130 is transmitted to the threaded shaft 114 to force the threaded shaft 114 to rotate. This in turn will cause translation of the slider 116, tensioning of the control wire 118, and deflection of the tool 104 (not shown in FIGS. 3A and 3B).

Referring next to FIGS. 4A and 4B, if tension on the control wire 118 (not shown in FIG. 4) increases (as might occur if, for example, a stiff secondary tool is within the tool 104 or if the tool 104 is in contact with patient anatomy) and the torque applied to the knob assembly 106 is increased to above a threshold value (as might occur if, for example, a user attempts to force the rotation of the knob assembly 106 despite the tension on the control wire 118), the knob assembly 106 will decouple from the slide assembly 112, via the slip clutch assembly 120. More specifically, torque applied to the knob assembly 106 will cause rotation of the first catch 122; however, due to the tension on the control wire 118, the threaded shaft 114 will resist rotation. Due to the inclined side surfaces of the protrusions 128, the protrusions 128 will deflect radially outwardly from the axis of rotation upon contact with the protrusions 132, to clear the protrusions 132, and thereby release the grip of the first catch 122 on the second catch 124. Movement of the slide assembly 112 by rotation of the knob assembly 106 will thus be avoided, and further tensioning of the control wire 118 by rotation of the knob assembly 106 will be avoided. If tension on the control wire 118 persists and rotation the knob assembly 106 continues, the first catch 122 will continue to rotate, and the protrusions 132 will snap in and out of the slots between the protrusions 132 as the first catch 122 rotates.

Referring next to FIG. 5, if tension on the control wire 118 (not shown in FIG. 5) decreases (as might occur if, for example, the stiff secondary tool is removed from within the tool 104) and the torque applied to the knob assembly 106 is decreased to below the threshold value, the knob assembly 106 will recouple to the slide assembly 112 via the slip clutch assembly 120. More specifically, due to the resiliently flexible nature of the protrusions 128, as rotation of the first catch 122 is continued, the protrusions 128 will snap back into position between the protrusions 132, to again cause gripping of the second catch 124 by the first catch 122, and again cause rotation of the second catch 124 due to rotation of the first catch 122.

In the above examples, the slip clutch assembly 120 may optionally provide tactile and/or auditory feedback to a user, to indicate to the user that the control wire 118 is under tension. For example, the user may feel the snapping of the protrusions 128 into and out of the slots between the protrusions 132 as the first catch 122 rotates. Alternatively, or in addition, an audible or tactile click may be felt when the protrusions 128 snap in and out of the slots between the protrusions 132. Alternatively or in addition, the handle 102 may be provided with a visual indicator (e.g. a window that allows the user to see disengagement of the first catch 122 and the second catch 124) to indicate to the user that the control wire 118 is under tension.

In alternative examples, rather than providing only binary coupling and decoupling of the knob assembly 106 and the slide assembly 112, the slip clutch assembly 120 can be configured to provide intermediate levels of coupling. For example, the protrusions 128 and 132 can be configured so that for a range of torques applied to the knob assembly 106, the slip clutch assembly 120 transmits some rotation before decoupling the knob assembly 106 and the slide assembly 112. This can serve as an alerting system, to alert the user that the torque is approaching the threshold value.

In alternative examples, rather than or in addition to being used to control tension on the control wire 118, the slip clutch assembly 120 can be used to limit the amount of deflection that the user can impart to the tool 104.

In alternative examples, the slip clutch assembly 120 may be configured so that the resiliently flexible protrusions are coupled to the slide assembly 112, and the inflexible protrusions are coupled to the knob assembly 106.

In alternative examples, the first catch and second catch may grip each other with the use of magnets (i.e. magnetic forces) and/or frictional forces rather than or in addition to protrusions.

In alternative examples, the first catch and the second catch may include another number of protrusions (e.g. the first catch and the second catch can each include only a single protrusion).

In any of the above examples, a lubricant may be provided to facilitate movement of the various parts.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A steerable medical device comprising:
a handle having a rotatable knob assembly and housing a slide assembly, wherein the knob assembly is couplable to the slide assembly to drive movement of the slide assembly by rotation of the knob assembly;
an elongate tool extending from the handle;
at least one control wire coupled between the slide assembly and the tool, whereby movement of the slide assembly causes tensioning of the control wire, and tensioning of the control wire causes deflection of the tool; and
a first catch coupled to the knob assembly, and a second catch coupled to the slide assembly, whereby when torque applied to the knob assembly is below a threshold value, the first catch grips the second catch to drive movement of the slide assembly by rotation of the knob assembly, and whereby when torque applied to the knob assembly is above the threshold value, the first catch releases the grip on second catch to decouple the knob assembly from the slide assembly to avoid movement of the slide assembly by rotation of the knob assembly.

2. The steerable medical device of claim 1, wherein
the first catch comprises a first set of spaced apart protrusions arranged annularly around an axis of rotation and extending generally parallel to the axis of rotation; and
the second catch comprises a second set of spaced apart protrusions arranged annularly around the axis of rotation and extending generally parallel to the axis of rotation;
wherein the protrusions of the first set are removably receivable between the protrusions of the second set.

3. The steerable medical device of claim 2, wherein
the first set of protrusions extend from a first hub, and the first hub is fixed to the knob assembly and rotatable with the knob assembly; and
the second set of protrusions extend from a second hub, and the second hub is fixed to the slide assembly and rotatable with the slide assembly, and wherein the second hub comprises a shaft extending along the axis of rotation, and the shaft abuts the first hub.

4. The steerable medical device of claim 3, wherein when torque applied to the knob assembly is below the threshold value, the protrusions of the first set are received between the protrusions of the second set to grip the second catch and thereby drive movement of the slide assembly by rotation of the knob assembly.

5. The steerable medical device of claim 4, wherein when torque applied to the knob assembly is above the threshold value, the protrusions of the first set deflect to clear the protrusions of the second set and release the grip of the first catch on the second catch and avoid movement of the slide assembly by rotation of the knob assembly.

6. The steerable medical device of claim 5, wherein the protrusions deflect radially outwardly from the axis of rotation.

7. The steerable medical device of claim 5, wherein the protrusions of the first set and the protrusions of the second set comprise inclined side surfaces, to facilitate deflection of the protrusions of the first set.

8. The steerable medical device of claim 2, wherein the protrusions of the first set are in the form of resiliently flexible prongs, and the protrusions of the second set are in the form of inflexible bumps defining slots therebetween for receipt of the prongs.

9. A handle for a medical device, the handle comprising:
a rotatable knob assembly and a slide assembly, wherein the knob assembly is couplable to the slide assembly to drive movement of the slide assembly by rotation of the knob assembly;
a first catch coupled to the knob assembly, and a second catch coupled to the slide assembly, whereby when torque applied to the knob assembly is below a threshold value, the first catch grips the second catch to drive movement of the slide assembly by rotation of the knob assembly, and whereby when torque applied to the knob assembly is above the threshold value, the first catch releases the grip on second catch to decouple the knob assembly from the slide assembly to avoid movement of the slide assembly by rotation of the knob assembly.

10. The handle of claim 9, wherein
the first catch comprises a first set of spaced apart protrusions arranged annularly around an axis of rotation and extending generally parallel to the axis of rotation; and
the second catch comprises a second set of spaced apart protrusions arranged annularly around the axis of rotation and extending generally parallel to the axis of rotation;
wherein the protrusions of the first set are removably receivable between the protrusions of the second set.

11. The handle of claim 10, wherein the protrusions of the first set are in the form of resiliently flexible prongs, and the protrusions of the second set are in the form of inflexible bumps defining slots therebetween for receipt of the prongs.

12. The handle of claim 10, wherein
the first set of protrusions extends from a first hub, and the first hub is fixed to the knob assembly and rotatable with the knob assembly; and
the second set of protrusions extends from a second hub, and the second hub is fixed to the slide assembly and rotatable with the slide assembly.

13. The handle of claim 12, wherein the second hub comprises a shaft extending along the axis of rotation, and the shaft abuts the first hub.

14. The handle of claim 12, wherein when torque applied to the knob assembly is below the threshold value, the protrusions of the first set are received between the protrusions of the second set to grip the second catch and thereby drive movement of the slide assembly by rotation of the knob assembly.

15. The handle of claim 14, wherein when torque applied to the knob assembly is above the threshold value, the protrusions of the first set deflect radially outwardly from the axis of rotation to clear the protrusions of the second set and release the grip on the second catch and avoid movement of the slide assembly by rotation of the knob assembly.

16. The handle of claim 15, wherein the protrusions of the first set and the protrusions of the second set comprise inclined side surfaces, to facilitate deflection of the protrusions of the first set.

17. A method for operating a medical device, comprising:
a. with a first catch releasably gripping a second catch, and with the first catch coupled to a knob assembly and the second catch coupled to a slide assembly, applying torque to the knob assembly to rotate the knob assembly, wherein the first catch comprises a first set of spaced apart protrusions arranged annularly around an axis of rotation and extending generally parallel to the axis of rotation, the second catch comprises a second set of spaced apart protrusions arranged annularly around the axis of rotation and extending generally parallel to the axis of rotation, and the protrusions of the first set are removably receivable between the protrusions of the second set; and
b. transmitting rotation of the knob assembly to the first set of protrusions to force the first set of protrusions to rotate around the axis of rotation, and transmitting the rotation of the first set of protrusions to the second set of protrusions to force the second set of protrusions to rotate around the axis of rotation.

18. The method of claim 17, further comprising:
c. increasing the torque applied to the knob assembly to exceed a threshold value; and
d. as a result of step c., releasing the grip of the first catch on the second catch.

19. The method of claim 18, further comprising:
e. decreasing the torque applied to the knob assembly to fall below the threshold value; and
f. as a result of step e., reapplying the grip of the first catch on the second catch.

20. The method of claim 17, further comprising providing tactile and/or auditory feedback to indicate that the first catch has released the grip on the second catch.
